(19) United States
(12) Patent Application Publication
(10) Pub. No.: US 2026/0053625 A1
(43) Pub. Date: Feb. 26, 2026

(54) Polymer Composition for Maxillofacial Implants and Implants Made Therefrom

(71) Applicant: Celanese International Corporation, Irving, TX (US)

(72) Inventors: Victoria Black, Cincinnati, OH (US); Rainer Walkenhorst, Melle (DE); Richard Moore, Florence, KY (US)

(21) Appl. No.: 19/303,679

(22) Filed: Aug. 19, 2025

Related U.S. Application Data
(60) Provisional application No. 63/685,030, filed on Aug. 20, 2024.

(51) Int. Cl. A61F 2/28; C08F 110/02

(52) U.S. Cl. CPC A61F 2/2875; C08F 110/02; A61F 2002/2878; A61F 2002/2882; A61F 2002/2889; A61F 2310/00005; C08F 2500/01; C08F 2500/07; C08F 2500/12; C08F 2500/24

(57) ABSTRACT

A biocompatible polymer composition is disclosed comprised of high density polyethylene particles. The high density polyethylene particles when sintered into an article can promote vascular growth. For example, when molded into an article, the particles produce a porous structure particularly well suited for maxillofacial implants.

### RELATED APPLICATIONS

The present application is based upon and claims priority to U.S. Provisional Application Ser. No. 63/685,030, having a filing date of Aug. 20, 2024, which is incorporated herein by reference.

### BACKGROUND

High density polyethylene polymers, including very high molecular weight polyethylene polymers and ultrahigh molecular weight polyethylene polymers, can be produced under controlled process conditions that are well suited for producing orthopedic implants. The high density polyethylene polymer, for instance, can be produced in a manner such that the polymer is in conformity with various regulatory criteria, such as those listed in ASTM F648 and ISO 5834-1/-2, which is incorporated herein by reference. These polyethylene polymers have found great commercial success in forming orthopedic implants through compression molding or ram extrusion, such as hip or knee protheses.

The use of high density polyethylene polymers for producing maxillofacial implants, however, such as cranial implants or facial implants, has been met with some but limited success. Maxillofacial implants, for instance, typically require unique properties for use in patients. The implants, for instance, not only need to have a certain amount of porosity, but also should demonstrate excellent mechanical properties. Maxillofacial implants, for instance, should possess a pore structure and other qualities that promote bony ingrowth and implant flexibility which are requirements much different than when designing other orthopedic implants.

In view of the above, a need currently exists for a high density polyethylene polymer composition that is well suited to producing medical implants having a porous structure. A need also exists for a high density polyethylene polymer composition that is well suited to producing maxillofacial implants, including facial implants and cranial implants.

### SUMMARY

In general, the present disclosure is directed to a high density polyethylene polymer composition well suited for producing maxillofacial implants (e.g. craniomaxillofacial implants), including cranial implants and facial implants. In accordance with the present disclosure, the high density polyethylene polymer composition is comprised of particles having a large particle size distribution and including coarse particles. When molded or sintered into an article, the polymer composition of the present disclosure produces a porous structure that is well suited for facilitating vascular ingrowth. Articles formed from the polymer composition also have excellent mechanical properties that make them well suited for use in producing maxillofacial implants.

In one embodiment, for instance, the present disclosure is directed to a polymer composition for producing medical implants. The polymer composition comprises high density polyethylene particles. The particles have an average particle size (d50) of from about 300 microns to about 950 microns, such as from about 400 microns to about 850 microns. The high density polyethylene particles also have a particle size distribution such that the high density polyethylene particles include particles having a size of less than about 300 microns, such as less than about 250 microns, such as less than about 150 microns, and include particles having a size of greater than about 1,000 microns, such as greater than about 1,500 microns, such as greater than about 2,000 microns. The high density polyethylene polymer can have an average molecular weight of from about 300,000 g/mol to about 10,000,000 g/mol, such as from about 600,000 g/mol to about 10,000,000 g/mol, such as from about 700,000 g/mol to about 8,000,000 g/mol and can have a melt flow rate of less than about 10 g/10 min, such as less than about 3 g/10 min, such as less than about 2.5 g/10 min, such as less than about 2.0 g/10 min when tested at 190° C. and at a load of 21.6 kg according to ISO Test 1133.

In one aspect, the high density polyethylene particles can have a potato-like shape. For instance, the particles can have an aspect ratio of from about 1 to about 4. The high density polyethylene polymer can have a viscosity number of at least about 300 mL/g, such as at least about 400 mL/g, such as at least about 500 mL/g, such as at least about 600 mL/g when tested according to ASTM D4020.

The high density polyethylene particles can display a bulk density of greater than about 200 kg/m3 and less than about 490 kg/m³, such as greater than about 300 kg/m³ and less than about 480 kg/m³, such as greater than about 400 kg/m³ and less than about 470 kg/m³, such as greater than about 400 kg/m³ and less than about 455 kg/m³.

The polymer composition of the present disclosure can contain the high density polyethylene polymer in an amount greater than about 99.5% by weight, such as in an amount greater than about 99.9% by weight. The polymer composition can be free of stabilizers, processing aids, and/or antioxidants. In one aspect, the high density polyethylene polymer contains titanium in an amount less than about 150 ppm, such as in an amount less than about 100 ppm, such as in an amount less than about 40 ppm. The high density polyethylene polymer can contain aluminum in an amount less than about 100 ppm, such as in an amount less than about 20 ppm. The high density polyethylene polymer can contain calcium in an amount less than about 50 ppm, such as in an amount less than about 5 ppm. The high density polyethylene polymer can contain chlorine in an amount less than about 90 ppm, such as in an amount less than about 30 ppm. The high density polyethylene polymer can have a density of greater than about 920 kg/m³, such as greater than about 935 kg/m³, such as greater than about 940 kg/m³, such as greater than about 945 kg/m³ when tested according to ASTM Test D792 or D1505.

The present disclosure is also directed to implants made from the polymer composition as described above. In one aspect, the implants can be made through sintering to form a porous structure. Alternatively, the implant can be made through compression molding and/or 3D printing. In one aspect, the implant comprises a maxillofacial implant. For instance, the implant can comprise a cranial implant, a zygomatic implant, a mandibular angle implant, an orbital implant, or a subperiosteal implant. Implants made according to the present disclosure can have a porous structure. In one aspect, the implant can have an average pore size of from about 50 microns to about 500 microns, such as from about 75 microns to about 300 microns. The implants can display a porosity of at least about 30%, such as at least about 40%, such as at least about 50%, and generally less than about 90%, such as less than about 80%, such as less than about 70%.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is a perspective view of one embodiment of a maxillofacial implant made in accordance with the present disclosure; and

FIG. 2 is another embodiment of a maxillofacial implant made in accordance with the present disclosure.

Repeat use of reference characters in the present specification and drawings is intended to represent the same or analogous features or elements of the present invention.

### Definitions

The melt flow rate of a polymer or polymer composition is measured according to ISO Test 1133 at 190° C. and at a load of 21.6 kg.

Particle size including average particle size (d50) and particle size distribution is measured using laser diffraction/light scattering according to ISO Test 13320 (2020).

The average molecular weight of a polymer is determined using the Margolies' equation. Molecular weight can be determined by first measuring the viscosity number according to DIN EN ISO Test 1628.

Tensile modulus, tensile stress at yield, tensile strain at yield, tensile stress at 50% break, tensile stress at break, and tensile nominal strain at break are all measured according to ISO Test 527-2/1B.

As used herein, bulk density is measured untapped according to ISO 60 (2023).

The amount of titanium, aluminum, calcium, and chlorine contained in a polyethylene polymer is determined according to ASTM Test F648-14.

Polymer density is determined according to ASTM Test D792 or D1505.

Izod impact strength is measured according to ASTM Test F648-14, Annex A1.

### DETAILED DESCRIPTION

It is to be understood by one of ordinary skill in the art that the present discussion is a description of exemplary embodiments only and is not intended as limiting the broader aspects of the present disclosure.

In general, the present disclosure is directed to a biocompatible polymer composition well suited to producing medical implants, particularly maxillofacial implants. In accordance with the present disclosure, the polymer composition contains high density polyethylene polymer particles. The high density polyethylene polymer not only contains coarse or relatively large particles, but also contains relatively small particles. Thus, the polymer composition has a relatively broad particle size distribution. In addition to the particle size distribution, the polymer particles also have a morphology that, when molded into an implant, produce a porous article that has excellent mechanical properties. In this manner, implants made from the polymer composition not only promote bony ingrowth but also display flexibility with excellent strength characteristics.

Thus, the high density polyethylene polymer composition of the present disclosure possesses a particle size and particle size distribution that influences many properties of molded articles made from the composition, including porosity and flexibility. It is believed that the presence of the coarse particles produces a pore structure when molded that facilitates bony ingrowth and implant flexibility in many implant applications, including facial or cranial implants. The composition can be molded through sintering. In one aspect, the particles are sintered together without applying pressure. Pressure-less sintering of high-density polyethylene particles is a consolidation process where loose polymer powder is transformed into a porous body without the application of external pressure, relying instead on heat and surface forces to bond the particles together. The polymer particles can be heated to a temperature just above the polymer's softening or melting point, usually in a mold or oven during the process.

The high density polyethylene polymer composition can contain particles having a particle size of greater than about 800 microns, such as greater than about 1,000 microns, such as greater than about 1,200 microns, such as greater than about 1,500 microns, such as greater than about 1,700 microns, such as greater than about 2,000 microns. The high density polyethylene polymer composition can be formulated such that the composition does not contain any particles that have a size greater than about 5,000 microns, such as greater than about 4,000 microns, such as greater than about 3,000 microns, such as greater than about 2,500 microns, such as greater than about 2,100 microns. In one aspect, the high density polyethylene polymer composition can also be formulated such that the composition does not contain any particles that have a size greater than about 2000 microns, such as greater than about 850 microns, such as greater than about 600 microns, such as greater than about 425 microns, such as greater than about 370 microns, such as greater than about 250 microns.

The high density polyethylene polymer composition can also contain relatively small particles. For instance, the polymer composition can contain particles having a size of less than about 500 microns, such as less than about 400 microns, such as less than about 300 microns, such as less than about 250 microns, such as less than about 200 microns, such as less than about 175 microns, such as less than about 150 microns. In one aspect, the high density polyethylene polymer composition can be formulated such that the composition does not contain any particles that have a size less 1200 microns, such as less than about 600 microns, such as less than about 450 microns, such as less than about 250 microns, such as less than about 150 microns, such as less than about 100 microns, such as less than about 50 microns. In one aspect, the polymer composition can be formulated such that the composition contains a small fraction (less than about 0.5% by weight, such as less than about 0.1% by weight, such as less than about 0.01% by weight) of particles having a size smaller than about 100 microns, such as less than about 50 microns, such as less than about 10 microns, such as less than about 5 microns.

The high density polyethylene polymer composition can contain particles having an average particle size (d50) of from about 200 microns to about 1600 microns. The average particle size can be greater than about 225 microns, such as greater than about 300 microns, such as greater than about 350 microns, such as greater than about 400 microns, such as greater than about 450 microns, such as greater than about 500 microns, such as greater than about 550 microns, such as greater than about 600 microns, such as greater than about 700 microns, such as greater than about 800 microns, such as greater than about 900 microns, and less than about 1570 microns, such as less than about 950 microns, such as less than about 900 microns, such as less than about 850 microns, such as less than about 800 microns, such as less than about 750 microns, such as less than 700 microns, such as less than about 650 microns, such as less than about 600 microns, such as less than about 550 microns, such as less than about 500 microns, such as less than about 450 microns, such as less than about 400 microns, such as less than about 350 microns, such as less than about 300 microns

The high density polyethylene polymer composition can contain particles having a D10 particle size of from about 155 microns to about 1220 microns. The D10 particle size can be greater than about 225 microns, such as greater than about 300 microns, such as greater than about 400 microns, such as greater than about 425 microns, such as greater than about 500 microns, such as greater than about 600 microns, such as greater than about 700 microns, and less than about 1000 microns, such as less than about 800 microns, such as less than about 700 microns, such as less than about 300 microns.

The high density polyethylene polymer composition can contain particles having a D90 particle size of from about 300 microns to about 2500 microns. The D90 particle size, for example, can be greater than about 300 microns, such as greater than about 350 microns, such as greater than about 400 microns, such as greater than about 500 microns, such as greater than about 600 microns, such as greater than about 700 microns, and less than about 2200 microns, such as less than about 1500 microns, such as less than about 1300 microns, such as less than about 1100 microns, such as less than about 800 microns, such as less than about 600 microns.

The high density polyethylene polymer composition can contain particles having a span of from about 0.5 to about 1.3, such as from about 0.6 to about 0.9.

The high density polyethylene polymer used to form the polymer composition of the present disclosure can generally have a density of about 0.92 g/cm³ or greater, such as about 0.93 g/cm³ or greater, such as about 0.94 g/cm³ or greater, and generally less than about 1 g/cm³, such as less than about 0.97 g/cm³.

The high density polyethylene particles can display a bulk density of greater than about 200 kg/m³ and less than about 490 kg/m³, such as greater than about 300 kg/m³ and less than about 480 kg/m³, such as greater than about 400 kg/m³ and less than about 470 kg/m³, such as greater than about 400 kg/m³ and less than about 455 kg/m³.

The high density polyethylene polymer can be made from over 90% ethylene derived units, such as greater than 95% ethylene derived units, or from 100% ethylene derived units. The polyethylene can be a homopolymer or a copolymer, including a terpolymer, having other monomeric units. In one aspect, the high density polyethylene can be a high density polyethylene homopolymer according to the specification of ASTM Test D4020.

The high density polyethylene can be a high molecular weight polyethylene, a very high molecular weight polyethylene, and/or an ultrahigh molecular weight polyethylene. “High molecular weight polyethylene” refers to polyethylene compositions with an average molecular weight of at least about 3×10⁵ g/mol and, as used herein, is intended to include very-high molecular weight polyethylene and ultra-high molecular weight polyethylene. For purposes of the present specification, the molecular weights referenced herein are determined in accordance with the Margolies equation (“Margolies molecular weight”).

“Very-high molecular weight polyethylene” refers to polyethylene compositions with a weight average molecular weight of less than about 3×10⁶ g/mol and more than about 1×10⁶ g/mol. In some embodiments, the molecular weight of the very-high molecular weight polyethylene composition is between about 2×10⁶ g/mol and less than about 3×10⁶ g/mol.

“Ultra-high molecular weight polyethylene” refers to polyethylene compositions with an average molecular weight of at least about 3×10⁶ g/mol. In some embodiments, the molecular weight of the ultra-high molecular weight polyethylene composition is between about 3×10⁶ g/mol and about 30×10⁶ g/mol, or between about 3×10⁶ g/mol and about 20×10⁶ g/mol, or between about 3×10⁶ g/mol and about 10×10⁶ g/mol, or between about 3×10⁶ g/mol and about 6×10⁶ g/mol.

Although the high density polyethylene polymer can be a high molecular weight polyethylene, a very-high molecular weight polyethylene, or an ultra-high molecular weight polyethylene, in one embodiment, the high density polyethylene polymer has an average molecular weight of greater than about 300,000 g/mol, such as greater than about 400,000 g/mol, such as greater than about 500,000 g/mol, such as greater than about 600,000 g/mol, such as greater than about 650,000 g/mol, such as greater than about 700,000 g/mol, such as greater than about 750,000 g/mol, such as greater than about 800,000 g/mol, such as greater than about 900,000 g/mol, such as greater than about 1,000,000 g/mol, such as greater than about 1,500,000 g/mol. The molecular weight of the high density polyethylene polymer is generally less than about 12,000,000 g/mol, such as less than about 10,000,000 g/mol, such as less than about 8,000,000 g/mol, such as less than about 6,000,000 g/mol, such as less than about 5,000,000 g/mol, such as less than about 4,500,000 g/mol, such as less than about 3,500,000 g/mol, such as less than about 2,500,000 g/mol, such as less than about 1,500,000 g/mol, such as less than about 1,000,000 g/mol.

Any method known in the art can be utilized to synthesize the high density polyethylene polymer particles. The polyethylene powder is typically produced by the catalytic polymerization of ethylene monomer or optionally with one or more other 1-olefin co-monomers, the 1-olefin content in the final polymer being less or equal to 10% of the ethylene content, with a heterogeneous catalyst and an organo aluminum or magnesium compound as cocatalyst. The ethylene can be polymerized in gaseous phase or slurry phase at relatively low temperatures and pressures. The polymerization reaction may be carried out at a temperature of between 50° C. and 100° C. and pressures in the range of 0.02 and 2 MPa.

The molecular weight of the polyethylene can be adjusted by adding hydrogen. Altering the temperature and/or the type and concentration of the co-catalyst may also be used to fine tune the molecular weight.

Suitable catalyst systems include but are not limited to Ziegler-Natta type catalysts, metallocene catalysts, and/or post metallocene catalysts. Typically Ziegler-Natta type catalysts are derived by a combination of transition metal compounds of Groups 4 to 8 of the Periodic Table and alkyl or hydride derivatives of metals from Groups 1 to 3 of the Periodic Table. Transition metal derivatives used usually comprise the metal halides or esters or combinations thereof. Exemplary Ziegler-Natta catalysts include those based on the reaction products of organo aluminum or magnesium compounds, such as for example but not limited to aluminum or magnesium alkyls and titanium, vanadium or chromium halides or esters. The heterogeneous catalyst might be either unsupported or supported on porous fine grained materials, such as silica or magnesium chloride. Such support can be added during synthesis of the catalyst or may be obtained as a chemical reaction product of the catalyst synthesis itself.

In one embodiment, a suitable catalyst system could be obtained by the reaction of a titanium (IV) compound with a trialkyl aluminum compound in an inert organic solvent at temperatures in the range of −40° C. to 100° C., preferably −20° C. to 50° C. The concentrations of the starting materials are in the range of 0.1 to 9 mol/L, preferably 0.2 to 5 mol/L, for the titanium (IV) compound and in the range of 0.01 to 1 mol/L, preferably 0.02 to 0.2 mol/L for the trialkyl aluminum compound. The molar ratio of titanium and aluminum in the final mixture can be in the range of 1:0.01 to 1:4.

In another embodiment, a suitable catalyst system is obtained by a one or two-step reaction of a titanium (IV) compound with a trialkyl aluminum compound in an inert organic solvent at temperatures in the range of −40° C. to 200° C., preferably −20° C. to 150° C. In the first step, the titanium (IV) compound is reacted with the trialkyl aluminum compound at temperatures in the range of −40° C. to 100° C., preferably −20° C. to 50° C. using a molar ratio of titanium to aluminum in the range of 1:0.1 to 1:0.8. The concentrations of the starting materials are in the range of 0.1 to 9.1 mol/L, preferably 5 to 9.1 mol/L, for the titanium (IV) compound and in the range of 0.05 and 1 mol/L, preferably 0.1 to 0.9 mol/L for the trialkyl aluminum compound. The titanium component is added to the aluminum compound over a period of 0.1 min to 800 min, preferably 30 min to 600 min. In a second step, if applied, the reaction product obtained in the first step is treated with a trialkyl aluminum compound at temperatures in the range of −10° C. to 150° C., preferably 10° C. to 130° C. using a molar ratio of titanium to aluminum in the range of 1:0.01 to 1:5.

In yet another embodiment, a suitable catalyst system is obtained by a procedure wherein, in a first reaction stage, a magnesium alcoholate is reacted with a titanium chloride in an inert hydrocarbon at a temperature of 50° to 100° C. In a second reaction stage, the reaction mixture formed is subjected to heat treatment for a period of about 10 to 100 hours at a temperature of 110° to 200° C. accompanied by evolution of alkyl chloride until no further alkyl chloride is evolved, and the solid is then freed from soluble reaction products by washing several times with a hydrocarbon.

In a further embodiment, catalysts supported on silica, such as for example the commercially available catalyst system Sylopol 5917 can also be used.

In one embodiment, especially when producing a relatively high molecular weight polyethylene, a metallocene-type catalyst may be used. For example, any metallocene or single-site type catalyst can be used to produce the polyethylene polymer. The catalyst can be made from metals from groups 4 and/or 6. In one embodiment, two different metallocene-type catalysts can be used to produce the polyethylene polymer. For instance, the metallocene catalysts may be made from metals, such as hafnium and/or chromium.

Using such catalyst systems, the polymerization is normally carried out in suspension at low pressure and temperature in one or multiple steps, continuous or batch. The polymerization temperature is typically in the range of 30° C. to 130° C., preferably in the range of 50° C. and 90° C. and the ethylene partial pressure is typically less than 10 MPa, preferably 0.05 and 5 MPa. Aluminum compounds can be used as co-catalyst such that the ratio of Al:Ti (co-catalyst versus catalyst) is in the range of 0.01 to 100:1, more preferably in the range of 0.03 to 50:1. The solvent is an inert organic solvent as typically used for Ziegler type polymerizations. Examples are butane, pentane, hexane, heptane, cyclohexane, octane, nonane, decane, their isomers and mixtures thereof. The polymer molecular mass is controlled through feeding hydrogen. The ratio of hydrogen partial pressure to ethylene partial pressure is in the range of 0 to 50, preferably the range of 0 to 10. The polymer is isolated and dried. Salts of long chain fatty acids may be added as a stabilizer.

Generally a cocatalyst such as aluminoxane or alkyl aluminum or alkyl magnesium compound is also employed.

In one aspect, the high density polyethylene particles are produced in a manner that makes the polymer well suited for medical applications, such as for producing implants and prosthetics. For instance, in one embodiment, the high density polyethylene polymer is made such that the polymer conforms with the requirements of ASTM F648 (2014). For instance, the high density polyethylene polymer can be produced in a manner that contains a very low amount of impurities. For instance, the high density polyethylene polymer can contain titanium in an amount less than about 150 ppm, such as in an amount less than about 100 ppm, such as in an amount less than about 40 ppm. The high density polyethylene polymer can contain aluminum in an amount less than about 100 ppm, such as in an amount less than about 20 ppm. In addition, the high density polyethylene polymer can contain calcium in an amount less than about 50 ppm, such as in an amount less than about 5 ppm, and can contain chlorine in an amount less than about 90 ppm, such as in an amount less than about 30 ppm. Titanium, aluminum, calcium, and chlorine content can be determined according to ASTM F648.

In order to achieve the desired particle size distribution as described above, the polymerization process, in one embodiment, can be controlled so as to achieve desired particle sizes. Screening or sieving can be used in order to remove, isolate and then optionally combine particles with different sizes in a controlled manner. In one aspect, particles having different sizes can be produced in different polymerization processes and then can be combined. In still another embodiment, particles can be ground to a desired size and the sizes can be combined in a desired manner.

The shape of the high density polyethylene particles can vary depending upon the particular application. In one aspect, for instance, the particles can have a spherical shape or can have a multi-lobal shape. In one aspect, the particles are produced so as to have a potato-like shape, meaning that the particles are generally round but have a length dimension that is longer than a width dimension. For instance, the high density polyethylene particles can have an aspect ratio of greater than about 1, such as greater than about 1.2, such as greater than about 1.4, such as greater than about 1.6, such as greater than about 1.8, such as greater than about 2, and less than about 8, such as less than about 6, such as less than about 4, such as less than about 3.8, such as less than about 3.5, such as less than about 3.3, such as less than about 3, such as less than about 2.8.

The high density polyethylene polymer, in one embodiment, can display a viscosity number when tested according to ASTM D4020 (0.02%) of greater than about 300 mL/g, such as greater than about 500 mL/g, such as greater than about 600 mL/g, such as greater than about 1,000 mL/g. In one aspect, the high density polyethylene polymer can display a viscosity number of from about 500 mL/g to about 3,200 mL/g. In an alternative embodiment, the high density polyethylene polymer can display a viscosity number of greater than about 3,200 mL/g and less than about 10,000 mL/g, such as less than about 6,000 mL/g, such as less than about 5,000 mL/g.

The high density polyethylene polymer can also display excellent mechanical properties. For instance, when tested at 23° C. according to ASTM Test D638, Type IV, 1.5 mm±0.5 mm and at a rate of 5.08 cm/min, the polymer composition can display an ultimate tensile strength of greater than about 10 MPa, such as greater than about 20 MPa, such as greater than about 27 MPa, such as greater than about 35 MPa, such as greater than about 40 MPa, and less than about 60 MPa, and can display a yield of greater than about 19 MPa, such as greater than about 21 MPa, and less than about 35 MPa. The high density polyethylene polymer can also display excellent izod impact strength properties. For instance, when tested according to izod impact strength according to ASTM F648-14, Annex A1, the high density polyethylene polymer composition can display an impact strength of greater than about 5 kJ/m², such as greater than about 10 kJ/m², such as greater than about 20 kJ/m², such as greater than about 25 kJ/m², such as greater than about 73 kJ/m², such as greater than about 126 kJ/m², and less than about 200 kJ/m².

The high density polyethylene polymer composition can display an elongation of greater than about 250%, such as greater than about 340%, such as greater than about 380%, such as greater than about 500%, such as greater than about 600%, such as greater than about 700%, such as greater than about 800%, such as greater than about 900%, such as greater than about 1000%, and less than about 2,000%, such as less than about 1,800%, such as less than about 1,500%, such as less than about 1,200%, such as less than about 600% (ISO 527, part 1/2; test speed 50 mm/min).

When the polymer composition of the present disclosure is formulated to produce implants, the polymer composition, in one aspect, contains virtually no other components or ingredients except for the high density polyethylene polymer particles. For instance, the polymer composition can contain the high density polyethylene polymer in an amount greater than about 99% by weight, such as in an amount greater than about 99.3% by weight, such as in an amount greater than about 99.5% by weight, such as in an amount greater than about 99.8% by weight, such as in an amount greater than about 99.9% by weight. The polymer composition, for instance, can be free of stabilizers, processing aids, or antioxidants.

The high density polyethylene polymer generally has a relatively low melt flow rate. The melt flow rate, for instance, is generally less than about 10 g/10 min at 190° C. and at a load of 21.6 kg according to ISO Test 1133. In one aspect the melt flow rate can be from about 10 g/10 min to 0 g/10 min (not capable of being measured). For instance, the melt flow rate of the high density polyethylene polymer can be less than about 2.8 g/10 min, such as less than about 2.5 g/10 min, such as less than about 2.2 g/10 min, such as less than about 2 g/10 min, such as less than about 1.8 g/10 min, such as less than about 1.5 g/10 min, such as less than about 1 g/10 min, such as less than about 0.5 g/10 min, such as less than about 0.3 g/10 min, and can be 0 g/10 min, or generally greater than about 1 g/10 min, such as greater than about 1.3 g/10 min, such as greater than about 1.5 g/10 min, such as greater than about 1.8 g/10 min.

The high density polyethylene polymer composition of the present disclosure can be subjected to various different processes and techniques in order to form molded articles. In one embodiment, molded articles, such as implants, can be formed without crosslinking the high density polyethylene polymer. In an alternative embodiment, the high density polyethylene polymer can be crosslinked. Typically, an article is first formed and then the polymer is crosslinked.

The high density polyethylene polymer can be crosslinked using any suitable method. For instance, a chemical crosslinking agent can be incorporated into the polymer or the polymer particles and can later be exposed to irradiation. For example, the high density polyethylene particles, once formed into an article, can be crosslinked using either x-rays or gamma rays or e-beam irradiation through a process called radiation crosslinking. This process involves exposing the polymer to high-energy radiation which causes the formation of free radicals within the polymer chains. These free radicals then initiate crosslinking reactions leading to the formation of a three-dimensional network within the polymer particles.

For example, in one embodiment, the high density polyethylene particles or molded article can be exposed to either e-beam radiation, x-rays or gamma rays from a radiation source. E-beam radiation, x-rays and gamma rays are forms of ionizing radiation that have sufficient energy to penetrate the material and induce chemical reactions within the polymer chains. When the radiation interacts with the polymer, it generates the free radicals within the polymer chains by breaking chemical bonds. These free radicals are highly reactive species with unpaired electrons.

The free radicals produced during radiation exposure initiate crosslinking reactions between neighboring polymer chains. This process involves the formation of covalent bonds between polymer molecules, leading to the creation of a three-dimensional network structure. After the desired level of crosslinking is achieved, optionally, the polymer particles or article may undergo post-treatment steps such as cooling or annealing.

The amount of radiation used to crosslink the high density polyethylene can vary. When using X-ray radiation or e-beam radiation, for example, the polymer particles can be exposed to greater than about 40 kGy, such as greater than about 100 kGy, such as greater than about 150 kGy, such as greater than about 200 kGy, such as greater than about 250 kGy, and less than about 1,000 kGy, such as less than about 500 kGy. When using gamma radiation, the polymer particles can be exposed to greater than about 40 kGy, such as greater than about 100 kGy, such as greater than about 150 kGy, such as greater than about 200 kGy, such as greater than about 250 kGy, and less than about 1,000 kGy, such as less than about 500 kGy, such as less than about 300 kGy.

The polymer composition of the present disclosure can be molded into articles using various methods. For instance, the high density polyethylene particles can be molded into an article using compression molding, three dimensional printing, laser sintering, or the like. In one embodiment, porous articles are made according to the present disclosure according to a sintering process in which the particles are fused together without applying pressure.

For example, porous articles may be formed by a free sintering process which involves introducing the polyethylene polymer powder described above into either a partially or totally confined space, e.g., a mold, and subjecting the molding powder to heat sufficient to cause the polyethylene particles to soften, expand and contact one another. The mold can be made of steel, aluminum or other metals.

The mold can be heated in a convection oven, hydraulic press or infrared heater to a sintering temperature between about 140° C. and about 300° C., such as between about 160° C. and about 300° C., for example between about 170° C. and about 240° C. to sinter the polymer particles. The heating time and temperature vary and depend upon the mass of the mold and the geometry of the molded article. However, the heating time typically lies within the range of about 10 to about 100 minutes. During sintering, the surface of individual polymer particles fuse at their contact points forming a porous structure. Subsequently, the mold is cooled and the porous article removed. In general, a molding pressure is not required. However, in cases requiring porosity adjustment, a proportional low pressure can be applied to the powder.

Porous substrates made according to the present disclosure can have a porosity of greater than about 20%, such as greater than about 35%, such as greater than about 40%, such as greater than about 45%, such as greater than about 50%. The porosity is generally less than about 80%, such as less than about 60%, such as less than about 55%. Porosity can be determined according to DIN 66133. Average pore size which can also be determined according to DIN 66133 can generally be from about 15 microns to about 800 microns, such as from about 50 microns to about 800 microns. For instance, the average pore size can be greater than about 50 microns, such as greater than about 100 microns, such as greater than about 150 microns, such as greater than about 200 microns, such as greater than about 250 microns, such as greater than about 300 microns, such as greater than about 350 microns, such as greater than about 400 microns, such as greater than about 450 microns, such as greater than about 500 microns, and less than about 700 microns, such as less than about 600 microns, such as less than about 500 microns, such as less than about 400 microns. In one aspect, the average pore size can be from about 75 microns to about 300 microns. In an alternative embodiment, the average pore size can be from about 150 microns to about 600 microns.

Once the porous article is formed, the article can optionally be subjected to various post treatments if desired. For instance, in one embodiment, the porous article can be subjected to heat treatment which may increase the strength of the article. The surface of the porous article can also optionally be subjected to a treatment for creating a hydrophilic surface. For instance, the article can be exposed to a plasma treatment and/or can be subjected to a chemical treatment in which hydrophilic groups are attached to the surface of the polymer. Alternatively, a hydrophilic coating can be applied to the surface of the article.

When formed into an implant, prior to being inserted into the body of a patient, the article can be sterilized. In one application, for instance, the molded implant can be subjected to a gamma sterilization treatment which can be carried out, for instance, at an energy level of from about 20 kGy to about 50 kGy. Alternatively, the implant can be sterilized using ethylene oxide, an e-beam sterilization process or a plasma sterilization process.

All different types of implants can be molded in accordance with the present disclosure. Implants that can be made according to the present disclosure, for instance, include implants for use in the cranial, facial, hand or foot areas of a patient.

In one embodiment, the polymer composition of the present disclosure can be used to produce maxillofacial implants. Such implants can include cranial implants, zygomatic implants, mandibular angle implants, orbital implants, subperiosteal implants, and the like.

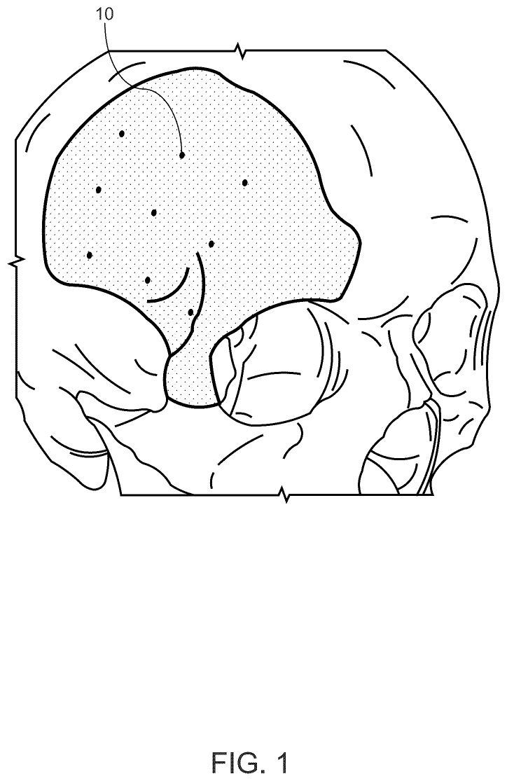

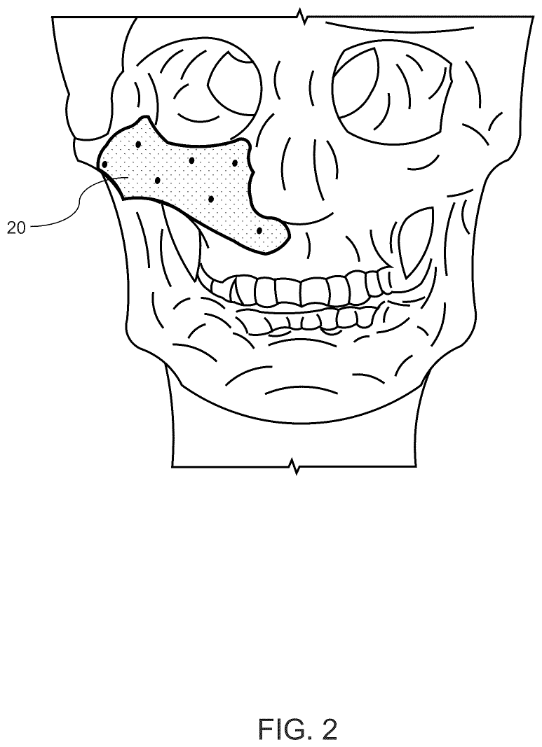

Referring to FIGS. 1 and 2, for instance, various embodiments of maxillofacial implants are shown. In FIG. 1, for instance, a cranial implant 10 made in accordance with the present disclosure is shown that has been inserted on the cranium of a patient. In FIG. 2, a facial implant 20 is illustrated that also can be used in accordance with the present disclosure. As described above, the porous nature of the implant allows for rapid bony ingrowth. In addition, the high density polyethylene polymer is relatively flexible and can be formed into any suitable shape.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A polymer composition for producing medical implants comprising:
high density polyethylene particles, the particles having an average particle size (d50) of from about 300 microns to about 950 microns, and wherein the particles have a particle size distribution such that the high density polyethylene includes particles having a size less than about 300 microns and includes particles having a size of greater than about 1,000 microns, the high density polyethylene polymer having an average molecular weight of from about 300,000 g/mol to about 10,000,000 g/mol, the high density polyethylene having a melt flow rate of less than about 10 g/10 min at 190° C. and at a load of 21.6 kg when tested according to ISO Test 1133.

2. A polymer composition as defined in claim 1, wherein the high density polyethylene particles display a bulk density of greater than about 200 kg/m³ and less than about 455 kg/m³.

3. A polymer composition as defined in claim 1, wherein no antioxidants have been added to the polymer composition.

4. A polymer composition as defined in claim 1, wherein the high density polyethylene polymer has an average molecular weight of from about 600,000 g/mol to about 10,000,000 g/mol and the high density polyethylene has a melt flow rate of less than about 3 g/10 min at 190° C. and at a load of 21.6 kg when tested according to ISO Test 1133.

5. A polymer composition as defined in claim 1, wherein the high density polyethylene particles have an average particle size (d50) of from about 400 microns to about 850 microns.

6. A polymer composition as defined in claim 1, wherein the high density polyethylene includes particles having a size of less than about 200 microns and includes particles having a size of greater than about 1,500 microns and have a melt flow rate of less than about 3 g/10 min at 190° C. and at a load of 21.6 kg when tested according to ISO Test 1133.

7. A polymer composition as defined in claim 1, wherein the high density polyethylene polymer displays a melt flow rate of less than about 2.5 g/10 min.

8. A polymer composition as defined in claim 1, wherein the polymer composition contains the high density polyethylene particles in an amount of about 99.5% by weight or greater.

9. A polymer composition as defined in claim 1, wherein the high density polyethylene particles have an aspect ratio of from about 1 to about 4.

10. A polymer composition as defined in claim 1, wherein the high density polyethylene polymer displays a viscosity number of at least about 300 mL/g when tested according to ASTM Test D4020.

11. A polymer composition as defined in claim 1, wherein the high density polyethylene polymer contains titanium in an amount of less than about 150 ppm, contains aluminum in an amount less than about 100 ppm, contains calcium in an amount less than about 50 ppm, and contains chlorine in an amount less than about 90 ppm.

12. A polymer composition as defined in claim 1, wherein no stabilizers or processing aids have been added to the polymer composition.

13. A polymer composition as defined in claim 1, wherein the high density polyethylene polymer displays a density of greater than about 920 kg/m³ when tested according to ASTM Test D792 or D1505.

14. A polymer composition as defined in claim 1, wherein the high density polyethylene polymer is biocompatible.

15. An implant formed from the polymer composition as defined in claim 1.

16. An implant as defined in claim 15, wherein the implant comprises a sintered article.

17. An implant as defined in claim 15, wherein the implant comprises a maxillofacial implant.

18. An implant as defined in claim 15, wherein the implant is porous.

19. An implant as defined in claim 18, wherein the implant has an average pore size of from about 50 microns to about 500 microns.

20. An implant as defined in claim 18, wherein the implant has a porosity of at least about 30%.

21. An implant as defined in claim 15, wherein the implant comprises a cranial implant.

22. An implant as defined in claim 15, wherein the implant comprises a zygomatic implant, a mandibular angle implant, an orbital implant, or a subperiosteal implant.
